(19) United States
(12) Patent Application Publication
(10) Pub. No.: US 2013/0331282 A1
(43) Pub. Date: Dec. 12, 2013

(54) MARKER COMPOSITION COMPRISING S1P PROTEIN FOR PREDICTION OF RISK IN OSTEOPOROTIC FRACTURE AND OSTEOPOROSIS

(71) Applicant: THE ASAN FOUNDATION, Seoul (KR)

(72) Inventors: Jung Min Koh, Seoul (KR); Ghi Su Kim, Seoul (KR); Seung Hun Lee, Gyeonggi-do (KR); Beom Jun Kim, Seoul (KR)

(21) Appl. No.: 13/865,688

(22) Filed: Apr. 18, 2013

(30) Foreign Application Priority Data
Jun. 8, 2012 (KR) 10-2012-0061678

(51) Int. Cl. G01N 33/92; C07K 16/18; C07K 14/47

(52) U.S. Cl. CPC G01N 33/92; C07K 14/47; C07K 16/18
USPC 506/9; 530/359; 530/389.3; 530/388.25; 530/387.3; 436/501; 435/7.93; 435/6.12; 435/6.11

(57) ABSTRACT

Disclosed herein is a marker composition that includes sphingosine 1-phosphate (S1P) protein and that may be used to predict the risk of fracture or osteoporosis; a kit that includes an antibody that specifically binds to the S1P protein and that can be used to predict the risk of fracture or osteoporosis; and a method that can be used to obtain information that allows prediction of the risk of fracture or osteoporosis and includes measurements of S1P protein concentrations by measuring the binding of a S1P-specific antibody to S1P protein. The S1P protein disclosed herein is highly expressed in individuals with fracture, regardless of their bone mineral density. Accordingly, it may be useful as a biomarker of the risk of fracture or osteoporosis.

### CROSS-REFERENCE TO RELATED APPLICATIONS

This application claims priority under 35 U.S.C. §119 to Korean Patent Application No. 10-2012-0061678 filed on Jun. 8, 2012 in the Korean Intellectual Property Office, the disclosure of which is incorporated herein by reference in its entirety.

### TECHNICAL FIELD

The present disclosure relates to a marker composition comprising sphingosine 1-phosphate (S1P) protein that is useful for predicting the risk of fracture or osteoporosis; a kit that includes an S1P-specific antibody for predicting the risk of fracture or osteoporosis; and a method that provides the information needed to predict the risk of fracture or osteoporosis, where S1P protein expression is determined by measuring the binding of an S1P-specific antibody to S1P.

### BACKGROUND

Osteoporosis refers to a state that bone mineral density is reduced due to lower amounts of the minerals and matrices that form bones. The result is bone fragility. Osteoporosis is the most frequently occurring metabolic bone disease (MBD) that is characterized by low bone mineral density (BMD) and an increased risk of fracture (Peacock, M., et al., Endocr. Rev. 23:303-326, 2002; Akhter, M. P., et al., Bone. 41(1):111-6, 2007). Increasing numbers of patients are currently being hospitalized because of osteoporotic fractures (Fogarty, P., et al., Maturitas. 52 Suppl 1:S3-6, 2005; Palacios, S., et al., Maturitas. 15;52 Suppl 1:S53-60. Review, 2005). Osteoporosis develops particularly frequently after menopause in women over the age of 40. Senile osteoporosis occurs in men and women over the age of 70.

Osteoporosis is currently diagnosed by a physical method such as X-ray scanning, but such methods require large diagnostic devices. Such methods are also hampered by the fact that they cannot predict further reductions in BMD. It is also difficult to accurately predict the risk of osteoporotic fracture on the basis of the BMD alone. Moreover, devices such as X-ray scans are associated with radiation exposure-associated safety problems.

Such limitations reveal the need for a quick, simple and accurate method that can detect various types of osteoporosis, such as postmenopausal osteoporosis and senile osteoporosis, early in the disease process and can be used to predict the risk of fracture.

To meet this need, the inventors conducted extensive studies to identify a marker that can be used to predict the risk of fracture or osteoporosis. These studies revealed that sphingosine 1-phosphate (S1P) is highly expressed in individuals with fractures, regardless of their BMD values. The inventors then completed the invention.

### REFERENCES OF THE RELATED ART

### Patent Document

Korean Patent Publication No.10-2010-0053302

### SUMMARY

An objective of the present disclosure is to provide a marker composition that comprises sphingosine 1-phosphate (S1P) protein and can be used to predict the risk of fracture or osteoporosis.

Another objective of the present disclosure is to provide a kit that includes an antibody that specifically binds to the S1P protein and can be used to predict the risk of fracture or osteoporosis.

Another objective of the present disclosure is to provide a method for measuring the binding of an S1P-specific antibody to the S1P proteins in biological samples. This method thus determines the levels of S1P protein in biological samples and thereby provides information that allows the risk of fracture or osteoporosis to be predicted.

To accomplish the objectives, the present disclosure provides a marker composition that comprises sphingosine 1-phosphate (SIP) protein and can be used to predict the risk of fracture or osteoporosis.

Further, the present disclosure provides a kit that includes an S1P-specific antibody and that can be used to predict the risk of fracture or osteoporosis.

Further, the present disclosure provides a method for measuring the binding of an S1P-specific antibody to the S1P proteins in biological samples. This method determines the levels of S1P protein in biological samples and thereby provides information that allows the risk of fracture or osteoporosis to be predicted.

The S1P protein disclosed herein is highly expressed in individuals with fracture, regardless of their BMD values. Accordingly, it may be useful as a biomarker that predicts the fracture or risk of osteoporosis occurrence.

### BRIEF DESCRIPTION OF THE DRAWINGS

The above and other objectives, features and advantages of the present disclosure will become apparent from the following description of certain exemplary embodiments given in conjunction with the accompanying drawings, in which:

FIGS. 1 and 2 depict the results of analyses that assess the relationship between blood S1P concentration and osteoporotic vertebral fracture; and

FIG. 3 depicts the relationship between S1P concentrations in the bone marrow and osteoporotic hip fracture. A and B depict this relationship before and after adjustment for potential confounders (age and sex).

### DETAILED DESCRIPTION OF EMBODIMENTS

Hereinafter, the present disclosure will be described in detail.

The present disclosure provides a marker composition comprising sphingosine 1-phosphate (S1P) protein, which can be used to predict the risk of fracture or osteoporosis.

In this disclosure, the term “marker” refers to any substance that can be used to predict the risk of fracture or osteoporosis because their levels in individuals who are at risk of fracture or osteoporosis are higher than their levels in normal individuals. These substances include organic biological molecules such as polypeptides or nucleic acids (e.g., mRNA), lipids, glycolipids, glycoproteins, and saccharides (monosaccharides, disaccharides, oligosaccharides, etc.).

The S1P protein disclosed herein is highly expressed in individuals with fracture, regardless of their bone mineral density. Thus, it may be useful as a biomarker for predicting the risk of fracture and osteoporosis.

Further, the present disclosure provides a kit that includes an S1P-specific antibody that can be used to predict the risk of fracture or osteoporosis.

In this disclosure, the term “antibody” refers to a protein molecule that binds to a specific antigenic site on another protein. For the purpose of this disclosure, the term “antibody” refers to antibodies that bind specifically to marker proteins. These include polyclonal antibodies, monoclonal antibodies, and recombinant antibodies.

Once the marker protein that can be used to predict the risk of fracture or osteoporosis is identified, as explained above, it is relatively easy to prepare a marker protein-binding antibody by using well-known technologies in the art.

Polyclonal antibodies can be produced by injecting the above-mentioned marker protein antigen (which can be used to predict the risk of fracture or osteoporosis) into an animal, after which the blood containing the antibodies is collected from the animal. The methods used to do this are well-known in the art. Polyclonal antibodies can be produced from any animal host, including goats, rabbits, sheep, monkeys, horses, pigs, cattle, and dogs.

Monoclonal antibodies can be produced by a well-known hybridoma method (Kohler and Milstein (1976), European Journal of Immunology 6:511-519) or a well-known phage antibody library method (Clackson et al., Nature, 352:624-628, 1991; Marks et al., J. Mol. Biol., 222:58, 1-597, 1991). An antibody produced by the above-mentioned methods can be isolated and purified by using gel electrophoresis, dialysis, salt precipitation, ion exchange chromatography, and affinity chromatography etc.

The antibody of the present disclosure is not only a complete antibody (it has two full-length light chains and two full-length heavy chains), it also bears the functional fragments of the antibody molecule. The term “functional fragments of the antibody molecule” refers to the fragments that, at a minimum, have an antigen-binding function. These fragments include Fab, F(ab′), F(ab′)₂, and Fv.

The kit of the present disclosure is composed of at least one element that is suitable for an analytic method, solution or device.

For example, the kit of the present disclosure may be a kit that includes an element that is essential for performing an enzyme-linked immunosorbent assay (ELISA). The ELISA kit may include an antibody that is specific for a marker protein and an agent that can be used to measure the level of the protein. The ELISA kit may include a reagent that can detect an attached antibody, such as a labeled secondary antibody, chromophores, an enzyme (e.g., an enzyme conjugated to antibody), and other substances that can bind to the substrate or antibody thereof. Further, it may include an antibody that is specific for a quantitative control group protein.

Further, the kit of the present disclosure may be a kit that includes the essential elements needed to perform polymerase chain reactions (PCR). These include genomic DNA that is derived from a sample that is to be analyzed, a primer set that is specific for the marker of the present disclosure, and the proper amount of DNA polymerase (for example, Taq-polymerase), deoxynucleotides (dNTP) mixture, PCR buffer, and water. The PCR buffer may include KCl, Tris-HCl, and MgCl₂. In addition, the kit of the present disclosure may include an element needed to perform the electrophoresis that is used to check the amplification of the PCR product.

Further, the kit of the present disclosure may be a kit that includes the essential elements needed to perform reverse transcription polymerase chain reaction (RT-PCR). The RT-PCR kit may include marker gene-specific primer pairs and may also include test tubes or other appropriate containers, reaction buffer solution (with varying pH and magnesium concentration), dNTPs, enzymes such as Taq-polymerase and reverse transcriptase, DNase, RNase inhibitor, DEPC-water, and sterile water etc. Further, it may include primer pairs that are specific for genes that serve as a quantitative control group.

Further, the kit of the present disclosure may be a kit that includes an essential element needed to perform DNA chip analysis. The DNA chip kit may include a substrate to which a gene or cDNA that corresponds to a fragment of the gene is attached as a probe. The substrate may also bear a quantitative structural gene or cDNA that corresponds to a fragment of the quantitative structural gene.

Further, the kit disclosed herein may be a type of microarray kit that contains a substrate to which the marker of the present disclosure is fixed.

Further, the present disclosure provides a method that can be used to obtain information that allows the risk of fracture or osteoporosis to be predicted. This method involves the measurement of S1P protein levels in a biological sample by using an S1P1-specific antibody and then measuring the antigen-antibody binding reaction.

Further, the present disclosure provides a method that can be used to obtain information that allows the risk of fracture or osteoporosis to be predicted. This method involves the measurement of S1P gene mRNA levels in a biological sample.

In this disclosure, the term “biological sample” includes, but is not limited to, samples such as tissues, cells, whole blood, serum, plasma, saliva, sputum, marrow fluid, and urine.

In this disclosure, the term “sphingosine 1-phosphate (S1P) level” means the S1P level that is determined by measuring the amount of mRNA or protein.

In this disclosure, the term “measuring mRNA level” refers to a process that measures the S1P-encoding mRNA in a biological sample, thereby allowing the risk of fracture or osteoporosis to be predicted. The methods that are suitable for the above-mentioned purpose may include any method known in the art. These include, but are not limited to PCR, RT-PCR, competitive RT-PCR, Real-time RT-PCR, RNase protection assay (RPA), and northern blotting and DNA chip technology.

In this disclosure, the term “measuring protein level” refers to a process that uses an S1P-specific antibody to measure the S1P protein in a biological sample, thereby allowing the risk of fracture or osteoporosis to be predicted. The methods that are suitable for the above-mentioned purpose may include any method known in the art. This includes, but is not limited to, western blot, ELISA, radioimmunoassay (RIA), radioimmunodiffusion, Ouchterlony immunodiffusion, rocket immunoelectrophoresis, immunohistochemical staining, immunoprecipitation assay, complement fixation assay, flow cytometry (Fluorescence Activated Cell Sorter, FACS), and protein chip assays.

The S1P protein disclosed herein is highly expressed in individuals with fracture, regardless of their bone mineral density. Accordingly, it may be useful as a biomarker for predicting the risk of fracture or osteoporosis.

The examples and experiments will now be described. The following examples and experiments are for illustrative purposes only; they are not intended to limit the scope of this disclosure.

### EXAMPLE 1

### A Case Control Study of the Serum S1P Concentrations in Healthy Postmenopausal Women with and Without Vertebral Fracture

(1) Selection of Test Candidates and Measurement of Key Variables

Healthy postmenopausal women who visited the Asan Medical Center (Seoul, Korea) because they were concerned about the possibility of osteoporosis or because they had been diagnosed with osteoporosis in a medical examination were enrolled. Menopause was defined as the absence of menstruation for at least 1 year. The presence of menopause was confirmed by measuring the serum follicle-stimulating hormone (FSH) concentration in the blood. The women who had entered menopause prematurely (aged <40 years), who had taken drugs that could affect bone metabolism for more than 6 months in the previous 12 months, or who had any disease that could affect bone metabolism were excluded. Finally, 460 women were selected. Of these, 69 were diagnosed with osteoporotic vertebral fracture and were placed in the case group. To perform a case control study, 69 women who were similar to the cases in terms of age (differing by no more than 2.5 years) and body mass index (BMI) (differing by no more than 1.0 kg/m²) were selected randomly from the remaining 391 women who did not have vertebral fracture. These women served as the control group. This study was approved by the Institutional Review Board of Asan Medical Center and was performed after receiving written informed consent from all candidates.

The bone mineral density (BMD, g/cm²) in the lumbar spine (L1-L4), femur neck, total femur, trochanter, and Ward's triangle of the proximal femur was measured by using dual energy X-ray absorptiometry (DXA; Lunar; Prodigy, Madison, Wis., USA).

Blood calcium and phosphorous concentrations were measured by using a cresolphthalein complexone and a phosphomolybdate ultraviolet method, respectively, with a Toshiba 200FR analyzer (Toshiba Medical Systems Co., Ltd, Tokyo, Japan). Bone turnover markers (BTMs) in the fasting blood and urine samples were also measured, as follows. Urinary N-terminal telopeptide of type I collagen (NTX) was measured by using an ELISA kit (Osteomark, Ostex International, Inc., Princeton, N.J., USA), and the results were standardized in proportion to urine creatinine concentration (nM BCE/mM creatinine). Serum bone-specific ALP (BSALP) concentration was measured by using a MetraTM BAP immunoassay kit (Quidel Corp., San Diego, Calif., USA). Serum osteocalcin (OSC) and C-terminal telopeptide of the type I collagen (CTX) concentrations were measured by using electrochemical luminescence immunoassays (Roche Diagnostics GmbH, Mannheim, Germany).

The results of these analyses are listed in Table 1.

TABLE 1

| Potential | Test group | | Control group | | |
|---|---|---|---|---|---|
| confounders | (n = 69) | | (n = 69) | | P-value |
| Age | 65.1 ± 6.7 | | 64.9 ± 6.5 | | 0.817 |
| Body weight (kg) | 57.4 ± 7.2 | | 55.4 ± 7.8 | | 0.123 |
| Height (cm) | 153.7 ± 6.0 | | 154.0 ± 5.4 | | 0.716 |
| BMI (kg/m²) | 24.2 ± 2.7 | | 23.4 ± 3.3 | | 0.075 |
| Number who smoke (%) | 1 | (1.4) | 4 | (5.8) | 0.172 |
| Number who drink ≧3 | 7 | (10.1) | 5 | (7.2) | 0.546 |
| alcohol U/day (%) | | | | | |
| Number who | 32 | (46.4) | 25 | (36.2) | 0.226 |
| exercise ≧30 min/day (%) | | | | | |

| | | | |
|---|---|---|---|
| Bone mineral density | | | |
| (g/cm²) | | | |
| Lumbar spine | 0.797 ± 0.140 | 0.877 ± 0.117 | 0.001 |
| Femur neck | 0.702 ± 0.109 | 0.738 ± 0.096 | 0.041 |
| Total femur | 0.750 ± 0.123 | 0.806 ± 0.098 | 0.004 |
| Trochanter | 0.589 ± 0.118 | 0.628 ± 0.100 | 0.041 |
| Shaft | 0.899 ± 0.152 | 0.979 ± 0.119 | 0.001 |
| Ward | 0.479 ± 0.114 | 0.523 ± 0.107 | 0.021 |
| Calcium (mg/dL)* | 9.0 ± 0.4 | 9.0 ± 0.3 | 0.702 |
| Phosphorus (mg/dL) | 3.9 ± 0.4 | 3.9 ± 0.5 | 0.941 |
| Bone turnover | | | |
| marker | | | |
| BSALP (U/L) | 30.6 ± 11.6 | 32.6 ± 15.2 | 0.396 |
| OSC (ng/mL) | 27.6 ± 11.9 | 32.4 ± 17.3 | 0.084 |
| NTX (nM BCE/mM) | 62.1 ± 42.2 | 58.8 ± 26.6 | 0.641 |
| CTX (ng/mL) | 0.559 ± 0.279 | 0.594 ± 0.270 | 0.518 |

As seen in Table 1, the test and control groups were 65.1±6.7 (47-77) and 64.9±6.5 (49-79) years old, respectively, and did not differ significantly in terms of blood calcium or phosphorous levels, BTM levels, body weight, height, BMI, or behavior factors. However, compared to the control group, the test group had significantly lower BMD values in all tested bones.

(2) Measurement of Blood S1P Protein Concentration

Fasting blood samples from all test and control candidates described in Example 1-(1) were centrifuged and their supernatants were isolated. The S1P protein concentration in the supernatant was measured three times by using a S1P competitive ELISA kit (Echelon Biosciences Inc., Salt Lake, Utah, USA), where 0.06 μmol/L was the minimum value. Table 2 indicates the mean SIP protein levels in the sera of the test and control groups before and after adjustment for age, BMI, recent smoking, drinking, and regular exercise. Table 2 also shows the mean S1P levels after further adjustment for lumbar spine bone mineral density (LS BMD).

TABLE 2

| | Test group | Control group | |
|---|---|---|---|
| | (n = 69) | (n = 69) | |
| | *Estimated mean S1P (95% CI) | | P-value |
| Unadjusted | 7.49 (6.82-8.16) | 5.58 (4.90-6.25) | <0.001 |
| data | | | |
| After | 7.54 (6.86-8.23) | 5.53 (4.84-6.21) | <0.001 |
| multivariable | | | |
| adjustment | | | |
| After further | 7.36 (6.67-8.06) | 5.65 (4.96-6.36) | 0.001 |
| adjustment for | | | |
| LSBMD | | | |

As seen in Table 2, the test group had higher blood S1P protein concentrations before and after adjustment for age, BMI, recent smoking, drinking, and regular exercise than the control group. Notably, this significant difference continued to persist even after further adjustment for lumbar spine BMD.

(3) Analysis of the Correlation Between Blood S1P Protein Concentration and BMD and Bone Turnover Markers (BTMs)

Pearson's correlation analysis was used to assess the correlation between blood S1P protein concentrations and BMD and bone turnover markers. The results are listed in Table 3.

TABLE 3

| | | P-value of | | P-value of |
|---|---|---|---|---|
| | | correlation | | correlation |
| BMD and | | analysis | | analysis |
| BTMs | | without | | with |
| variables | γ | adjustment* | γ | adjustment* |
| Bone mineral | | | | |
| density | | | | |
| (g/cm*) | | | | |
| Lumbar spine | −0.192 | 0.026 | −0.214 | 0.015 |
| Femur neck | −0.136 | 0.113 | −0.175 | 0.044 |
| Total femur | −0.150 | 0.080 | −0.182 | 0.036 |
| Trochanter | −0.131 | 0.124 | −0.166 | 0.056 |
| Shaft | −0.167 | 0.060 | −0.184 | 0.041 |
| Ward | −0.159 | 0.062 | −0.201 | 0.020 |
| Bone turnover | | | | |
| marker | | | | |
| BSALP (U/L) | 0.072 | 0.438 | 0.072 | 0.449 |
| OSC (ng/mL) | −0.076 | 0.413 | −0.067 | 0.480 |
| NTX (nM BCE/mM) | 0.241 | 0.014 | 0.242 | 0.016 |
| CTX (ng/mL) | 0.153 | 0.115 | 0.162 | 0.103 |

*Adjustment was for age, BMI, recent smoking, drinking, and regular exercise.

Table 3 shows that after adjustment for potential confounders, the blood S1P protein concentration correlated negatively with the BMD values in the lumbar spine (L1-L4), femur neck, total femur, femur shaft, and Ward's triangle. However, while the BMD value in the trochanter also showed a negative correlation with blood S1P protein levels, this correlation did not achieve statistical significance. Furthermore, after adjustment for potential confounders, only the bone resorption marker NTX correlated significantly with blood S1P protein levels. The bone formation markers BSALP and OSC did not correlate with blood S1P protein concentration.

(4) Analysis of the Correlation Between Blood S1P Protein Concentration and Vertebral Osteoporotic Fractures

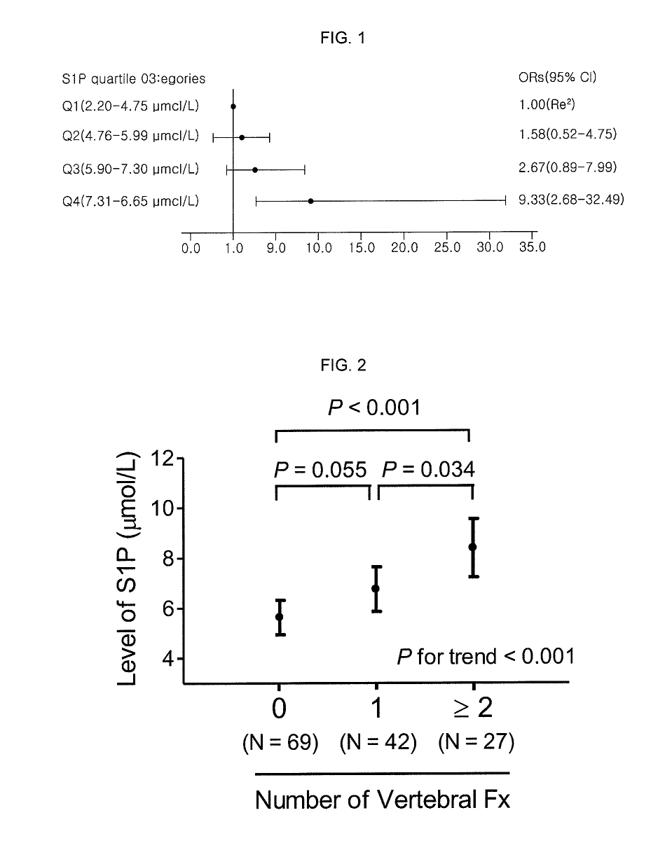

The blood S1P protein levels in subjects with and without osteoporotic vertebral fracture were analyzed further, as shown by FIG. 1 and FIG. 2.

In FIG. 1, the 138 subjects in the case control study were divided into quartiles on the basis of their S1P protein concentrations (shown on the y-axis) and the prevalence of vertebral osteoporotic fracture in each quartile was calculated. In the lowest SIP quartile category (Q1), 32.4% of the subjects had vertebral fractures. This prevalence rose steadily as the S1P concentrations rose: in Q2, Q3, and the highest S1P protein quartile category Q4, the prevalences were 38.2%, 52.8%, and 76.5%, respectively.

Multiple logistic regression analysis was then performed to determine the odds ratios (ORs) of vertebral osteoporotic fracture in the Q2, Q3, and Q4 groups; the Q1 group served as the reference group. While the ORs for Q2 and Q3 were not significant (see the right-hand dataset in FIG. 1), the highest quartile category (Q4) had a significant OR of 9.33 (95% CI=2.68-32.49). The OR of Q4 continued to be significant before and after adjustment for potential confounders (OR=6.80, 95% CI=2.33-19.81 before adjustment, and OR=9.12, 95% CI=2.85-29.23 after adjustment).

(5) Relationship Between Blood S1P Protein Concentration and Number of Vertebral Osteoporotic Fractures

FIG. 2 shows the serum S1P protein concentrations in the 138 subjects when they were divided into three groups depending on whether they had no (n=69), one (n=42), or two or more (n=27) vertebral fractures. While the subjects with one fracture did not differ significantly from the control subjects in terms of S1P protein levels (p=0.055), the subjects with two or more fractures differed significantly from both the controls (p<0.001) and the subjects with one fracture (p=0.034). The upward trend in S1P protein levels as the number of fractures rose was significant (p<0.001).

Thus, blood S1P protein concentration may be useful for predicting the risk of fracture, regardless of BMD values. To further test the value of S1P protein concentration as a marker for predicting the risk of fracture or osteoporosis, the following experiment was performed.

### EXAMPLE 2

### A Cohort Study of the S1P Protein Concentrations in Patients with or without Osteoporotic Hip Fracture Who were Scheduled for Hip Replacement

(1) Selection of Test Candidates

Sixteen patients who were scheduled to undergo hip replacement surgery at the orthopedics department of Asan Medical Center were enrolled. Patients who took drugs that could affect bone metabolism (e.g., osteoporosis drugs) for more than 6 months in the preceding 12 months and patients who had any disease that could cause secondary osteoporosis (e.g., hyperthyroidism) were excluded. Osteoporotic hip fracture was defined as fracture that was not caused by high impact, such as in an automobile accident. Of the 16 patients, four had osteoporotic hip fracture. This study was approved by the Institutional Review Board of Asan Medical Center and was performed after receiving written informed consent from all candidates.

(2) Measurement of Bone Marrow Fluid S1P Protein Concentration

The sex, age, body weight, height, and diagnosis at the time of surgery were recorded. The BMI was calculated on the basis of the body weight and height. Bone marrow was collected during the operation and was aliquoted into 200 μL aliquots and stored in a −70° C. deep freezer. To measure the S1P protein concentration (mmol/L) in the bone marrow fluid, an aliquot of bone marrow was centrifuged at 3,000 rpm, 4° C. for 5 min, and the marrow fluid was harvested and stored at −70° C. The S1P protein concentration in the fluid was then measured twice by using a S1P competitive ELISA kit (Echelon Biosciences Inc., Salt Lake City, Utah), where 0.06 μmol/L was the minimum value. Its coefficients of variation in the measurement and between the measurements were 6.4% and 6.1%, respectively. The mean of the two marrow fluid S1P protein concentration replicates measured in each patient was used for analysis. Serum calcium (mmol/L) and phosphorous concentrations (mmol/L) were measured by using the cresolphthalein comlexone and phosphomolybdate UV methods, respectively, with a Toshiba 200FR Autoanalyzer (Toshiba Medical Systems Co., Ltd., Tokyo, Japan). The glomerular filtration rate (mL/min/1.73 m²) was calculated by using the Cockroft-Gault formula. All coefficients of variation were less than 3.5%.

(3) Measurement of BMD

The BMD (g/cm²) in the lumbar spine and the proximal femur (the femur neck and the total femur) was measured by dual energy X-ray absorptiometry (Lunar; Prodigy, Madison, Wis.). The coefficients of variation of lumbar spine and femur neck BMDs were 0.67% and 1.25%, respectively.

(4) Statistical Analysis

The mean values of the patients with and without osteoporotic hip fracture were compared by using independent sample t-tests. The values were adjusted for confounders by Analysis of Covariance (ANCOVA). The confounders used were age and sex. The correlation between bone marrow S1P protein concentration and BMD was analyzed by partial correlation analysis after adjustment for the confounders, and three analysis models were established. Age and sex were included in model 1, body weight and height were included in model 2, and serum calcium concentration, phosphorous concentration and glomerular filtration rate were included in model 3. In every statistical analysis, P<0.05 was considered to indicate statistical significance.

(5) Analysis of the Correlation Between Blood S1P Protein Concentration and Osteoporotic Fracture

Of the 16 patients, nine (56%) were female. By contrast, of the four patients with osteoporotic hip fracture, three were female (75%). Of the 12 patients who underwent surgery for causes other than osteoporotic hip fracture, seven, four, and one underwent surgery because of osteoarthritis, avascular necrosis of the femoral head, and hip dislocation, respectively. The patients with osteoporotic hip fracture were 71.3±21.7 years old and their BMI was 20.3±3.9 kg/m². The control group patients were slightly but not significantly younger (64.9±9.8 years; P=0.607) and had a marginally significant higher BMI (25.1±3.9 kg/m²; P=0.050).

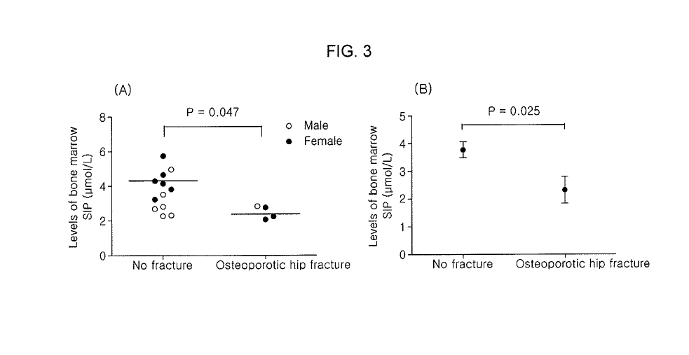

The mean marrow fluid S1P protein concentration of all 16 patients was 3.40±1.10 μmol/L (range: 2.08-5.74 μmol/L). As shown in FIG. 3A, the marrow fluid S1P protein concentrations of the patients with and without osteoporotic hip fracture were 2.48±0.38 and 3.70±1.09 μmol/L, respectively. Thus, the patients with osteoporotic hip fracture had significantly lower marrow fluid S1P protein concentrations than the control patients (P=0.047). After adjustment for age and sex, the difference between the two groups increased further: the estimated marrow fluid S1P protein concentrations of the patients with and without osteoporotic hip fracture were 2.31±1.94 and 3.76±1.10 μmol/L, respectively (P=0.025) (FIG. 3B). Analysis of the correlation between marrow fluid S1P protein concentrations and BMD failed to detect a significant correlation after adjustment for various confounders.

The data above indicate that patients with osteoporotic hip fracture have significantly lower marrow fluid S1P protein concentrations than control patients, which suggests that increased levels of S1P protein in the bone marrow have beneficial effects on bone metabolism.

Example 1 showed that higher serum S1P protein concentrations are associated with lower BMD, higher bone resorption, and a higher risk of osteoporotic fracture. On the contrary, in Example 2, it was confirmed that higher marrow fluid S1P concentration is associated with lower risk of osteoporotic hip fracture. Thus, it seems a higher osteoporotic fracture risk associates not only with higher peripheral blood S1P protein concentrations but also with lower marrow fluid S1P protein concentrations. This suggests that S1P protein does not increase bone resorption by directly affecting bone cells. It also suggests that both marrow S1P protein concentrations and the difference between S1P protein concentrations in the peripheral blood and the bone marrow can serve as markers of bone resorption.

The difference between the blood and bone marrow in terms of S1P protein concentrations is due to the fact that serum S1P protein concentrations are maintained at high levels by the continuous secretion of S1P protein by the endothelial cells of blood vessels, red blood cells, and platelets. By contrast, the S1P protein concentrations in tissues, including the bone marrow, are maintained at low levels by continuous degradation in cells by S1P lyase or S1P phosphatase. In peripheral blood with high S1P protein concentrations, osteoclast precursors predominantly express S1P receptor type 2 (S1PR2). The binding of S1P protein to S1PR2 activates the Rho signaling pathway in osteoclasts via G12/13 protein, which in turn promotes the movement of the osteoclast precursors to the bone marrow. Once in the bone marrow, the osteoclast precursors differentiate into mature osteoclasts, attach to the bone surface, and start to resorb bone. Thus, larger differences between the peripheral blood and the bone marrow in terms of S1P protein concentrations promote the movement of osteoclast precursors to the bone surface, which increases bone resorption and thereby raises the risk of fracture.

These observations show that the bone marrow SIP protein concentration and the difference between the bone marrow and peripheral blood concentrations of S1P protein may be useful as markers of the risk of fracture or osteoporosis.

While the present disclosure has been described with respect to the specific embodiments, it will be apparent to those skilled in the art that various changes and modifications may be made without departing from the spirit and scope of the disclosure as defined in the following claims.

What is claimed is:

1. A marker composition comprised of sphingosine 1-phosphate (S1P) protein can be used to predict the risk of fracture or osteoporosis.

2. The marker composition described in claim 1 can be used to predict the risk of fracture or osteoporosis by measurements of S1P protein concentration in the blood or bone marrow fluid.

3. A kit comprising an S1P protein-specific antibody can be used to predict the risk of fracture or osteoporosis.

4. The kit described in claim 3 can predict the risk of fracture or osteoporosis by measuring the S1P protein concentration in blood or marrow fluid.

5. The kit described in claim 3 that can predict the risk of fracture or osteoporosis may be based on a polyclonal, monoclonal, or recombinant antibody.

6. A method to obtain information that allows prediction of the risk of fracture or osteoporosis may estimate the sphingosine 1-phosphate (S1P) protein concentration in biological samples by measuring the binding of an S1P-specific antibody to the S1P antigen.

7. A method to obtain information that allows prediction of the risk of fracture or osteoporosis may estimate the sphingosine 1-phosphate (S1P) gene mRNA concentrations in a biological sample.

8. The method to obtain information that allows prediction of the risk of fracture or osteoporosis and is described in claim 6 comprises measurements of S1P protein concentrations in biological samples and assessments of the correlation between the measured value and the presence of fracture or osteoporosis, wherein the biological sample is at least one of the following: tissues, cells, whole blood, serum, plasma, saliva, sputum, bone marrow fluid, and urine.

9. The method to obtain information that allows prediction of the risk of fracture or osteoporosis and is described in claim 7 comprises measurements of the S1P gene mRNA concentration in biological samples and assessments of the correlation between the measured value and the presence of osteoporotic fracture or osteoporosis, wherein the biological sample is at least one of the following: tissues, cells, whole blood, serum, plasma, saliva, sputum, marrow fluid, and urine.

10. In the method that is described in claim 6 and is used to obtain information that allows prediction of the risk of fracture or osteoporosis, the protein level is measured by at least one of the following methods: western blot, enzyme-linked immunosorbent assay (ELISA), radioimmunoassay (RIA), radioimmunodiffusion, Ouchterlony immunodiffusion, rocket immunoelectrophoresis, immunohistochemical staining, immunoprecipitation assay, complement fixation assay, flow cytometry (Fluorescence Activated Cell Sorter, FACS), and protein chip assays.

11. In the method that is used to obtain information that allows prediction of the risk of fracture or osteoporosis and is described in claim 7, the mRNA expression level is measured by at least one of the following methods: polymerase chain reaction (PCR), reverse transcription polymerase chain reaction (RT-PCR), competitive reverse transcription polymerase chain reaction (Competitive RT-PCR), real-time reverse transcription polymerase chain reaction (Real-time RT-PCR), RNase protection assay (RPA), northern blotting, and DNA chip analysis.
